(19) United States
(12) Patent Application Publication
(10) Pub. No.: US 2004/0219117 A1
(43) Pub. Date: Nov. 4, 2004

(54) Method of spray-coating deodorant, antibacterial and antifungal agent and deodorized and antibacterially and antifungally treated articles

(75) Inventor: Osamu Shibata, Hyogo (JP)

Correspondence Address: BACON & THOMAS, PLLC, 625 SLATERS LANE, FOURTH FLOOR, ALEXANDRIA, VA, 22314

(73) Assignee: Home Taste Co., Ltd., Kobe-shi (JP)

(21) Appl. No.: 10/764,540

(22) Filed: Jan. 27, 2004

(30) Foreign Application Priority Data
Feb. 25, 2003 (CN) 03107559.2

(51) Int. Cl. A61K 7/32; A61K 33/42

(52) U.S. Cl. 424/65; 424/601

(57) ABSTRACT

The present invention provides a method of spray coating deodorant, antibacterial and antifungal agent which can exert a deodorant, antibacterial and antifungal effect even in a dark place shielded from light and which is not hazardous to animals, human and environment as well as a deodorized and antibacterially and antifungally treated article. An article to be subjected to a deodorizing, antibacterial and antifungal treatment is spray-coated with a solution comprising as an active component a titanium phosphate-based compound or a condensate thereof at a coating rate of 10 to 30 g/m² using a spray gun whose pumping rate is 33 to 34 cc per minute.

### BACKGROUND OF THE INVENTION

1. Field of the Invention

The present invention relates to a method of spray coating deodorant, antibacterial and antifungal agent which can exert a deodorant, antibacterial and antifungal effects even in a dark place shielded from light and which is not hazardous to animals, human and environment as well as a deodorized and antibacterially and antifungally treated article.

2. Description of the Prior Art

A titanium oxide is employed usually as a deodorant agent, antibacterial agent or antifungal agent for furniture or household products.

However, the titanium oxide, when present alone, can not exert an active effect in the absence of a light (UV light), while it can exert the effect in the presence of the light when combined with silver or gold, which may cause an adverse effect on environment or human life. IN addition, the titanium oxide requires a binder when it is supported on a carrier article.

### SUMMARY OF THE INVENTION

An objective of the invention is to provide a method of spray coating deodorant, antibacterial and antifungal agent which can exert deodorant, antibacterial and antifungal effects even in a dark place shielded from light without adversely affecting environment or human and without requiring any binder and also to provide a deodorized and antibacterially and antifungally treated article.

A method of spray coating deodorant, antibacterial and antifungal agent according to the invention is a method comprising spray-coating a solution comprising as an active component a titanium phosphate-based compound represented by Formula: Ti(OH)ₓ(PO₄)_{y}(HPO₄)_{z}(H₂PO₄)₁(OR)ₘ wherein R is an alkyl group having 1 to 4 carbon atoms, each of x, y, z, l and m is an integer of 0 or more and x+3y+2z+l+m=4 or a condensate thereof onto an article to be subjected to a deodorizing, antibacterial and antifungal treatment at a coating rate of 10 to 30 g/m² using a spray gun whose pumping rate is 33 to 34 cc per minute.

A deodorant, antibacterial and antifungal agent containing a titanium phosphate-based compound described above or a condensate thereof as an active component is not hazardous to animals, human and environment and exerts a deodorant, antibacterial and antifungal effects even in a dark place shielded from light by means of a reaction with oxygen contained in air. In addition, it requires no binder and exhibits a sustained deodorant, antibacterial and antifungal effects without undergoing any deterioration.

As a result of the spray-coating using spray gun described above, the outer surface of an article to be coated uniformly with a film of a deodorant, antibacterial and antifungal agent.

A deodorized and antibacterially and antifungally treated article according to the invention has a film of a deodorant, antibacterial and antifungal agent formed on its outer surface as a result of the spray coating described above.

### DETAILED DESCRIPTION OF THE PREFERRED EMBODIMENT

A deodorant, antibacterial and antifungal agent used in an inventive spray coating method contains as an active component a titanium phosphate-based compound represented by Formula:

Ti(OH)ₓ(PO₄)_{y}(HPO₄)_{z}(H₂PO₄)₁(OR)ₘ,

wherein R is an alkyl group having 1 to 4 carbon atoms, each of x, y, z, l and m is an integer of 0 or more and x+3y+2z+l+m=4 or a condensate thereof.

R is an alkyl group having 1 to 4 carbon atoms, and a too small number of carbon atoms leads to a reduced viscosity of the solution, which gives a too thin film which may lead to a reduced deodorant, antibacterial and antifungal effect, while a too large number of carbon atoms leads to an increased viscosity of the solution, which gives a too thick film which may readily be peeled off. R is preferably an ethyl group or an isopropyl group.

Such a titanium phosphate-based compound may for example be Ti(OH) (H₂PO₄)₂(OR), Ti(OH) (PO₄), Ti(OH)₂(H₂PO₄) (OR), Ti(OH) (HPO₄) (OR), Ti(OH) (HPO₄) (H₂PO₄), Ti(OH)₂(H₂PO₄)₂, Ti(OH)₃(H₂PO₄), Ti(OH)₃(OR) and the like.

A titanium phosphate-based compound or its condensate can be obtained by reacting titanium tetrachloride with water or an alcohol having 1 to 4 carbon atoms or a mixture thereof followed by a reaction with phosphoric acid.

The invention is characterized by spray coating a titanium phosphate-based compound or its condensate thus obtained onto an article to be subjected to a deodorizing, antibacterial and antifungal treatment in a manner described below.

A titanium phosphate-based compound or its condensate is diluted with water or an alcohol, and a resultant solution is spray-coated using a spray gun whose pumping rate is 33 to 34 cc per minute onto an article placed on a table at a shooting range of 40 to 45 cm at a coating rate of 10 to 30 g/m², more preferably 20 g/m², over a coating period of about 20 to 35 seconds, more preferably 30 seconds and then dried.

As a result of the spray coating using the spray gun described above, a deodorant, antibacterial and antifungal agent coating film whose dry thickness is 0.05 to 0.5 μm can be applied uniformly on the outer surface of the article. A dry film thickness less than 0.05 μm leads to a poor deodorant, antibacterial and antifungal effects, while that exceeding 0.5 μm allows the film to be peeled off readily, resulting in the loss of the deodorant, antibacterial and antifungal effects.

The present invention is effective especially in deodorizing formaldehyde which causes a so-called “sick-house” syndrome or hydrogen sulfide and the like. It is also possible to suppress the growth of a surrounding hazardous microorganism by caulking and achieve a substantial sterilization. Since no silver or gold is employed unlike to a prior art method, the safety is ensured without adversely affecting environment or human life. The invention is effective especially against coliforms, Staphylococcus aureus, coliform O157, methicillin-resistant Staphylococcus aureus (MRSA) as well as most of hazardous microorganisms. The film hardness is 9H as a pencil hardness, and a high hardness property is experienced as an excellent resistance to staining.

Articles which can be spray-coated are various furniture, household products, interior products, beds, mats, building materials and the like. Those exemplified typically are wooden cabinets, shelves, racks, tables, desks, chairs, beds, sofas, carpets, flooring, wallpapers, wall materials, ceiling materials and the like.

What is claimed is:

1. A method of spray coating deodorant, antibacterial and antifungal agent comprising spray-coating a solution comprising as an active component a titanium phosphate-based compound represented by Formula: Ti(OH)ₓ(PO₄)_{y}(HPO₄)_{z}(H₂PO₄)₁(OR)ₘ wherein R is an alkyl group having 1 to 4 carbon atoms, each of x, y, z, l and m is an integer of 0 or more and x+3y+2z+l+m=4 or a condensate thereof onto an article to be subjected to a deodorizing, antibacterial and antifungal treatment at a coating rate of 10 to 30 g/m² using a spray gun whose pumping rate is 33 to 34 cc per minute.

2. A deodorized and antibacterially and antifungally treated article having a deodorant, antibacterial and antifungal coating film thereon which is formed by spray-coating a solution comprising as an active component a titanium phosphate-based compound represented by Formula: Ti(OH)ₓ(PO₄)_{y}(HPO₄)_{z}(H₂PO₄)₁(OR)ₘ wherein R is an alkyl group having 1 to 4 carbon atoms, each of x, y, z, l and m is an integer of 0 or more and x+3y+2z+l+m=4 or a condensate thereof onto an article to be subjected to a deodorizing, antibacterial and antifungal treatment at a coating rate of 10 to 30 g/m² using a spray gun whose pumping rate is 33 to 34 cc per minutes.
